(19) United States
(12) Patent Application Publication
(10) Pub. No.: US 2010/0040680 A1
(43) Pub. Date: Feb. 18, 2010

(54) MULTIPARTICULATE SELECTIVE SEROTONIN AND NOREPINEPHRINE REUPTAKE INHIBITOR FORMULATION

(76) Inventors: Felix Lai, Hayward, CA (US); Richard Ting, San Ramon, CA (US); Christopher A. Simpson, San Jose, CA (US); Victoria Kellogg, Fremont, CA (US); Sibel Ucpinar, Mountain View, CA (US)

Correspondence Address: Martin Endres, Esq.;HEDMAN & CONSTIGAN, P.C., 1185 Avenue of the Americas, New York, NY, 10036-2601, US

(21) Appl. No.: 12/192,609

(22) Filed: Aug. 15, 2008

(51) Int. Cl. A61K 9/54; A61K 9/14; A61K 31/381; A61K 9/22; A61K 9/36; A61K 9/32

(52) U.S. Cl. 424/458; 424/490; 424/494; 424/497; 424/468

(57) ABSTRACT

A multiparticulate oral pharmaceutical composition that contains a plurality of delayed release coated selective serotonin and norepinephrine reuptake inhibitor particles.

### TECHNICAL FIELD

The present invention relates to a multiparticulate oral dosage form containing a selective serotonin and norepinephrine reuptake inhibitor (“SSNRI”). More specifically, the present invention relates to a multiparticulate dosage form for oral administration that comprises a plurality of delayed release particles. The preferred SSNRI is duloxetine, prodrugs, isomers, metabolites or pharmaceutically acceptable salts thereof. The present invention is useful in the treatment of depression, diabetic peripheral neuropathic pain, anxiety disorders, obsessive compulsive disorders, attention deficit disorders and fibromyalgia.

### BACKGROUND OF THE INVENTION

Psychotropic and psychopharmacologic agents are well known in the medical and pharmaceutical fields. These agents are useful in altering the mind and behavior. One well known and commonly prescribed class of psychotropic and psychopharmacologic agents is antidepressants. Antidepressants are useful in treating depression and related disorders such as anxiety and compulsive disorders. A genus of antidepressants that have been used in the treatment of depression and mental disorders are known as serotonin selective reuptake inhibitors (“SSRIs”). SSRIs are believed to prevent the reuptake of the neurotransmitter serotonin. Some of the more widely known and prescribed SSRIs are escitalopram, fluoxetine, paroxetine and sertraline.

SSRI oral dosage forms have been described in the art. For example, United States Published Patent Application No. 2007/0141150 discloses a multiparticulate fluoxetine hydrochloride dosage form. The dosage form contains enteric coated fluoxetine hydrochloride pellets. The pellets consist of a core of fluoxetine hydrochloride, a subcoating applied to the fluoxetine hydrochloride core and an enteric coating applied to the subcoating. The subcoating comprises a substance that rapidly reacts with the functional groups of the enteric coating to prevent reactions between the fluoxetine core and the enteric material. The substance in the subcoating that reacts with the functional groups of the enteric material is an amino acid, preferably glycine.

U.S. Pat. No. 6,548,084 discloses a number of controlled release SSRI formulations such as enteric coated tablets and hydrophilic matrix tablets that contain paroxetine. A multiparticulate encapsulated paroxetine product is also described in U.S. Pat. No. 6,548,084. This multiparticulate dosage form consists of beads that employ waxes and waxy materials as the release controlling agent.

Although the use of SSRIs has proven effective for some patients, a group of antidepressants that prevent the reuptake of both serotonin and norepinephrine are gaining acceptance. The dual reuptake inhibitors are sometimes referred to as SSNRIs or SNRIs. Examples of some SSNRIs currently in use are venlafaxine, desvenlafaxine, sibutramine, nefazodone, milnacipran, desipramine, duloxetine and bicifadine.

U.S. Pat. No. 6,274,171 discloses a multiparticulate venlafaxine hydrochloride dosage form. The dosage form employs extended release pellets consisting of core of venlafaxine hydrochloride, microcrystalline cellulose and hydroxypropyl methylcellulose. The core is coated with film coating consisting ethylcellulose and microcrystalline cellulose.

U.S. Pat. No. 5,508,276 discloses a multiparticulate duloxetine dosage form. The disclosed dosage forms consist of enteric coated pellets consisting of an inert core onto which is applied a layer consisting of duloxetine, a binder and a filler. The duloxetine layer is coated with an inert separating layer comprising a water soluble film forming polymer such as hydroxypropyl methylcellulose, hydroxypropyl cellulose or polyvinylpyrrolidone. The inert separating layer is subsequently coated with an enteric coating layer that comprises an enteric polymer and a water soluble plasticizer or a partially neutralized enteric polymer.

United States Published Patent Application No. 2006/0165776 also discloses multiparticulate duloxetine dosage forms. The dosage form described in United States Published Patent Application No. 2006/0165776 consists of a plurality of microtablets. Each microtablet consists of a duloxetine hydrochloride core prepared by granulating duloxetine hydrochloride, lactose and hydroxypropyl methylcellulose. The granules are mixed with hydrogenated castor oil and crospovidone and the resulting mixture is compressed into microtablets. The microtablets are coated with an intermediate layer consisting of hydroxypropyl methylcellulose, talc and titanium dioxide. The intermediate layer is further coated with an enteric layer comprising an enteric polymer, talc and a water soluble plasticizer.

United States Published Patent Application No. 2007/0292511 discloses another multiparticulate dosage form comprising duloxetine. The dosage form described in United States Published Patent Application No. 2007/0292511 comprises an inert core coated with a drug layer consisting of duloxetine hydrochloride, binders, coating agents, glidants and thickness agents. The drug layer is subsequently coated with a separating layer comprising water soluble coating agents, diluents and thickness agents. The separating layer is finally coated with an enteric layer consisting of an enteric polymer, a glidant and a water soluble polymer.

All the aforementioned multiparticulate SSNRI dosage forms employ very complicated multistage manufacturing processes and employ a number of excipients, especially in the drug layer or core and separating layer.

It is an object of the present invention to provide a stable oral multiparticulate dosage form for SSNRIs that is easy to manufacture and employs a few excipients.

It is a further object of the present invention to provide a stable oral multiparticulate dosage from for SSNRIs that employs a drug core, a separating layer applied to the drug core and an enteric coating applied to the separating layer wherein the separating layer is free of water insoluble materials, i.e. comprises only water soluble materials.

It is still a further object of the present invention to provide a stable oral a stable oral multiparticulate dosage from for SSNRIs that employs a drug core, a separating layer applied to the drug core and an enteric coating applied to the separating layer wherein the enteric coating is free of water soluble materials, i.e. comprises only water insoluble materials, very slightly soluble materials or slightly soluble materials.

These and other objects of the present invention will become apparent from a review of the appended specification.

### SUMMARY OF THE INVENTION

The present invention accomplishes the above objects and others by providing a novel dosage form for SSNRIs that comprises a drug core, a water soluble seal coating surrounding the drug core and a delayed release coating surrounding the seal coating.

The drug core may be prepared by layering the SSNRI onto an inert starting seed or by mixing the SSNRI with at least one pharmaceutically acceptable excipients such as a filler or diluent and subjecting the mixture to extrusion/spheronization techniques or compression to form microtablets.

The seal coating preferably comprises a low molecular weight water soluble polymer that will surround the SSNRI drug core. The seal coating may protect the SSNRI from potential adverse interactions with the delayed release coating materials (solids and solvents) during the application of the delayed release coating and during storage. The seal coating may also provide a surface, preferably a smooth and uniform surface, which aids in the application and adhesion of the subsequent delayed release coating.

The seal coating does not contain any SSNRI or other pharmaceutically active drug and is a separate and distinct layer between the drug core and the delayed release coating of the present invention. The seal coating should disintegrate or dissolve within 30 minutes, preferably in less than 20 minutes and most preferably in less than 15 minutes when it is exposed to an aqueous fluid environment. In one embodiment of the present invention, the seal coating comprises only water soluble materials. It is believed that the use of large quantities of water insoluble materials such as talc in the rapidly disintegrating coating can affect the release properties of the dosage form.

The delayed release coating is applied to the seal coating. The delayed release coating comprises at least one enteric material. An enteric material is a compound, preferably a polymer, which does not dissolve in low pH environments such as the stomach but will dissolve at pH level above 4.5, preferably at a pH above 5. The delayed release coating may further comprise conventional processing aids, such as plasticizers, anti-adherents, lubricants, surfactants, antifoaming agents, coloring agent and mixtures of the foregoing.

In one embodiment of the present invention the delayed release coating is free of any water soluble component and only comprises the enteric material and a slightly soluble, very slightly soluble and/or insoluble processing aids.

In another embodiment of the present invention, the delayed release coating employs a combination enteric materials that dissolve at different pH points.

### DETAILED DESCRIPTION OF THE INVENTION

The subject invention concerns a stable delayed release multiparticulate oral pharmaceutical formulation or dosage form comprising an SSNRI such as venlafaxine, desvenlafaxine, sibutramine, nefazodone, milnacipran, desipramine, duloxetine, bicifadine or pharmaceutically acceptable salts thereof. The preferred SSNRI is duloxetine and pharmaceutically acceptable salts thereof, in particular the hydrochloride salt of duloxetine.

The SSNRI is formulated into a multiparticulate solid oral dosage form in accordance with the present invention by mixing the SSNRI with conventional pharmaceutical excipients to prepare an SSNRI core. The SSNRI core is then coated with a seal coating. A delayed release coating is subsequently applied to the seal coating.

The final dosage form of the present invention can be a tablet, capsule or suitable container comprising the delayed release coated particles. A preferred embodiment of the present invention is a multiparticulate tablet or capsule that comprises a plurality, i.e more than one, of delayed release SSNRI particles prepared in accordance with the present invention. As used herein, the term “particle” includes small individual units that comprise at least the drug and one pharmaceutically acceptable excipient. The particles may range in size from about 0.1 mm to about 3.0 mm, preferably about 0.5 to about 1.5 mm. The particles may be coated granules, microtablets (sometimes referred to as mini tablets), beads or pellets.

In the case of granules, the granule may be formed by conventional wet or dry granulation techniques wherein the SSNRI is mixed with at least one pharmaceutically acceptable excipient such as a filler to create a granule that will be coated as described in detail below. The granule may be irregularly shaped or approximately spherical.

In the case of microtablets, the SSNRI is mixed with conventional pharmaceutical excipients and compressed into small tablets using conventional techniques such as a rotary tablet press. The compressed microtablets are coated as described below.

The particles may also be beads or pellets prepared by layering the SSNRI onto an inert starting seed such as a sugar seed, a microcrystalline cellulose seed, a plastic bead, a wax bead or a glass bead. The drug layered beads or pellets may also be coated as described below. The inert starting seeds should have a narrow particle size distribution. The inert starting seeds may range in size from 18 to 40 mesh, with preferred ranges being within 20-25 mesh or 30-35 mesh.

The bead or pellets may also be prepared by extrusion spheronization wherein the SSNRI and at least one pharmaceutically acceptable excipient are mixed and/or granulated. The resulting mixture is then extruded into small discrete units, spheronized and sized to acceptable size ranges. The spheronized SSNRI cores are then coated as described below.

The acceptable pharmaceutical excipients employed in the core can be any of the well known excipients describe in the Handbook of Pharmaceutical Excipients or the United States Pharmacopeia. Some of the preferred excipients are binders, fillers, lubricants and glidants.

Acceptable binders are, for example, celluloses such as hydroxypropyl methycellulose, hydroxypropyl cellulose and carboxymethycellulose sodium; polyvinylpyrrolidone; sugars; starches and other pharmaceutically acceptable substances with cohesive properties. A preferred type of binder is a film forming water soluble material.

Acceptable fillers, sometimes referred to as diluents, include sugars such as lactose, dextrose, sucrose, and maltose, calcium phosphate, kaolin, microcrystalline cellulose and the like.

Examples of anti-adherents, lubricants and glidants that may be used in the present invention include talc, magnesium stearate, calcium stearate, stearic acid, hydrogenated vegetable oils, polyethylene glycols, silicon dioxide and mixtures of the foregoing.

Once the SSNRI core is prepared, it is coated with a seal coating. The seal coating is applied to the SSNRI cores by conventional procedures such as the use of a coating pan or fluidized bed apparatus using water and/or conventional organic solvents for the coating solution. The materials for the seal coating are chosen from among pharmaceutically acceptable water soluble or water dispersible inert compounds or polymers. Examples of some of the materials useful for the seal coating include sugar, polyethylene glycol, povidone, polyvinyl alcohol, hydroxypropyl cellulose, methylcellulose, hydroxymethyl cellulose, hydroxypropyl methylcellulose, polyvinyl acetal diethylaminoacetate and the like. In one embodiment of the present invention the seal coating comprises only water soluble polymeric materials, preferably low viscosity water soluble polymers, i.e. less than 500 cps for a 2% aqueous solution at 20° C. and most preferably less than 100 cps for a 2% aqueous solution at 20° C.

The seal coating should also be free of any active pharmaceutical compound. The seal coating should also be free of any material that can react with the SSNRI in the core or the materials in the delayed release coating, especially the acidic functional groups of the enteric material employed in the delayed release coating.

In one embodiment of the present invention, the seal coating is a mixture of two water soluble polymer, preferably a mixture of water soluble polymers selected from the group consisting of hydroxypropyl methylcellulose, hydroxypropyl cellulose, polyvinylpyrrolidone, polyvinyl alcohol and polyethylene glycol. In a preferred embodiment, the seal coating is a mixture of hydroxypropyl methylcellulose and polyethylene glycol in a ratio of about 1:3 to 3:1, preferably about 1:2 to about 2:1 and most preferably about 1:1.

The seal coating should also be formulated so it rapidly dissolves or disintegrates when placed in an aqueous environment. One embodiment of the present invention the seal coating should dissolve or disintegrate within 30 minutes, preferably within 20 and most preferably within 15 minutes, when the seal coated SSNRI core is placed in 900 ml of dionized water.

The seal coating may also comprise other conventional processing aids known in the art such as a surfactant or antifoaming agent. The processing aids if employed should be inert to the materials in the SSNRI core and inert to the materials in the delayed release coating. The processing aids if employed in the seal coating should also be water soluble. Unless otherwise indicated, the term “water soluble” as used in this specification means approximately 35 parts of water or less are required to dissolve 1 part of the material.

After the seal coating is applied to the SSNRI core, a delayed release coating is applied to the seal coating.

The delayed release coating contains an enteric material. The enteric materials do not dissolve in acidic environments. Generally, the enteric materials do not dissolve until they encounter an aqueous media with a pH of about 4.5 or higher and preferably about 5.0 or higher. Representative examples of enteric material that can be used include cellulose acetate phthalate, hydroxypropylmethyl cellulose phthalate, polyvinyl acetate phthalate, carboxymethylethylcellulose, polymethacrylates such as Eudragit L (poly(methacrylic acid, methylmethacrylate), 1:1 ratio, MW (No. Av. 135,000—USP Type A) or Eudragit S (poly(methacrylic acid, methylmethacrylate, 1:2 ratio, MW (No. Av. 135,000—USP Type B), shellac, zein, alginates and mixtures thereof.

The delayed release coating may also include conventional processing aids such as fillers, diluents, antiadherents, lubricants and glidants described above as well as plasticizers. Suitable plasticizers include acetyl tributyl citrate, acetyl triethyl citrate, benzyl benzoate, castor oil, diacetylated monoglycerides, dibutyl phthalate, diethyl phthalate, dimethyl phthalate, dibutyl sebacate, glycerin, glycerol monostearate, polyethylene glycol, propylene glycol, triacetin, tributyl citrate, triethyl citrate and mixtures of the foregoing. A preferred class of plasticizers is the acetylalkyl citrates.

In one embodiment of the present invention, the delayed release coating comprises the enteric material and slightly soluble, very slightly soluble and/or insoluble processing aids. As used herein the term “slightly soluble” means approximately 100 to 1,000 parts of water are required to dissolve the processing aid. The term “very slightly soluble” means approximately 1,000 to 10,000 parts of water are required to dissolve 1 part of the processing aid and the term “insoluble” means more than 10,000 parts of water are required to dissolve 1 part of processing aid.

Examples of fillers or diluents which meet the slightly soluble, very slightly soluble or insoluble criteria include microcrystalline cellulose, kaolin, calcium carbonate, calcium phosphate, calcium sulfate, cellulose powdered and silicified microcrystalline cellulose.

Examples of the anti-adherents, lubricants, or glidants which meet the slightly soluble, very slightly soluble or insoluble criteria include talc, calcium stearate, magnesium stearate and colloidal silicon dioxide.

Examples of the plasticizers which meet the slightly soluble, very slightly soluble or insoluble criteria include acetyl tributyl citrate, acetyl triethyl citrate, benzyl benzoate, castor oil, diacetylated monoglycerides, dibutyl phthalate, diethyl phthalate, dimethyl phthalate, dibutyl sebacate, glycerol monostearate, tributyl citrate and mixtures of the foregoing.

In a preferred embodiment, the delayed release coating is free of any water soluble materials and comprises the enteric material and only very slightly soluble or insoluble processing aids, preferably only insoluble processing aids.

In an alternate embodiment, the delayed release coating comprises a mixture of two different enteric materials. The two different materials should dissolve or disperse at different pHs. For example one embodiment of the present invention may employ a mixture of enteric materials wherein one enteric material begins dissolving at a pH of about 5.0 and the other enteric material begins dissolving at a pH of 5.5 or higher. By adjusting the ratio of the two enteric materials and/or the thickness of the delayed release coating, it is believed the release of the SSNRI from the core can be controlled or sustained over a portion of the intestinal track and thereby avoid a large immediate release or dump of the SSNRI at one location in the intestinal tract. It is further believed the control or sustain aspect is due to a portion of the SSNRI core being exposed to the intestinal fluids when the first or lower pH enteric material dissolves or disintegrates while the second or higher pH enteric material remains intact. Preferably the ratio of low pH dissolving enteric material to high pH dissolving material is about 5:1 to about 1:1 and preferably about 4:1 to about 2:1.

The amount of enteric material employed may range from about 1% to about 25% of the total particulate weight, preferably about 5% to about 20% of the total particulate weight. It is believed that if the delayed release coating employs at least two different enteric materials, a thinner delayed release coating may be employed. If a combination of enteric materials is employed, the total amount of enteric material employed may range from about 1% to about 20% of the total particulate weight, preferably about 5% to about 15% of the total particulate weight.

After the delayed release coating is applied, a plurality of the delayed release particles can be filled into a hard or soft gelatin capsule or mixed with conventional tableting excipients and compressed into a tablet. The delayed release particles may also be filled into an acceptable container to allow the delayed release particles to be sprinkled onto food such as apple sauce for those individuals that have a difficult time swallowing a tablet of capsule.

The final dosage form of the present invention may also include an immediate release amount of the SSNRI. The immediate release component can be in the form of free SSNRI added to the capsule or tablet, particles of the SSNRI as described above that have not been coated with the delayed release coating or an immediate release layer of the SSNRI applied to the delayed release coating of the tablets or particles.

An embodiment of a dosage form in accordance with the present invention will have the following composition based upon the total weight of the delayed release particle:

| SNRI Core: | | | |
|---|---|---|---|
| SSNRI | 1.0-50% | (10-40% | preferred) |
| Inert carrier | 15-90% | (25-75% | preferred) |
| Binder | 0.5-10% | (1-5% | preferred) |
| Seal Coating | 0.1-10% | (0.5-5% | preferred) |
| Delayed Release Coating: | | | |
| Enteric Material | 1-25% | (5-20% | preferred) |
| plasticizer | 0-10% | (0.1-5% | preferred) |
| lubricant/glidant | 1-15% | (2-10% | preferred) |

### EXAMPLES OF THE INVENTION

The following are provided by way of example only and are by no means intended to be limiting.

### Example 1

A 60 mg controlled release multiparticulate duloxetine hydrochloride capsule in accordance with the present invention was prepared as follows:

### SSNRI Core

Approximately 16.16 kg of micronized duloxetine hydrochloride and 1.62 kg of hypromellose (Shin-Etsu's Pharmacoat #606) are dissolved in approximately 71 kg of purified water.

Approximately 30.22 kg of 20-25 mesh (710-840 micron) sugar spheres are loaded into a GPCG-30 fluidized bed coated. The duloxetine hydrochloride solution is then sprayed onto the sugar spheres under the following conditions:
product temperature: 26-47° C.;
atomization pressure: 1.0-1.5 bars;
air volume: 220-290 cfm;
spray rate: 50-240 g/min.

After the duloextine solution was consumed the SSNRI cores were dried in the fluidized bed apparatus for approximately 15 minutes at 60° C.

### Seal Coating

Approximately 0.60 kg of polyethylene glycol 6000 and 0.60 kg of hypromellose (Shin-Etsu's Pharmacoat #606) are dissolved in a mixture of 21.21 kg of alcohol USP and 1.14 kg of purified water to create a seal coating solution.

The seal coating solution is applied to the SSNRI cores prepared above using the same fluidized bed apparatus and the following conditions:
conditions:
product temperature: 25-40° C.;
atomization pressure: 1.0-1.5 bars;
air volume: 245-255 cfm;
spray rate: 75-125 g/min.

After the seal coating solution was consumed the seal coated SSNRI cores were dried in the fluidized bed apparatus for approximately 15 minutes at 45° C.

The dried seal coated cores were screened through 14 and 24 mesh screens and the seal coated cores retained on the 24 mesh screen were collected.

### Delayed Release Coating

A delayed release suspension comprising approximately 5.04 kg of hypromellose phthalate (Shin-Etsu's HP-50); 0.20 kg of acetyltributyl citrate NF; 2.31 kg of talc USP; 90.97 kg of acetone NF and 4.79 kg of purified water, USP was prepared. The delayed release suspension was applied to approximately 25.20 kg of the seal coated SSNRI cores using a fluidized bed apparatus under the following conditions:
product temperature: 22-26° C.;
atomization pressure: 1.5-2.0 bars;
air volume: 240-255 cfm;
spray rate: 75-190 g/min.

After the delayed release coating suspension was consumed the delayed release coated particles cores were dried in the fluidized bed apparatus for approximately 15 minutes at 45° C.

The dried delayed release coated particles were screened through 14 and 24 mesh screens and the seal coated cores retained on the 24 mesh screen were collected.

The screened delayed release coated particles were dusted with approximately 0.16 kg of talc and filled into hard gelatin capsules to create a multiparticulate dosage form containing approximately 60 mg of duloxetine. The final dosage form had the following composition:

| Ingredient | % (w/w) | mg/capsule |
|---|---|---|
| SSNRI CORE | | |
| Duloxetine Hydrochloride | 24.4 | 67.35 |
| Sugar Spheres | 45.6 | 125.9 |
| Hypromellose* | 2.4 | 6.75 |
| SEAL COATING | | |
| Polyethylene Glycol | 1.8 | 5.0 |
| Hypromellose* | 1.8 | 5.0 |
| DELAYED RELEASE COATING | | |
| Hypromellose Phthalate (HP-50) | 15.2 | 42.00 |
| Acetyltributyl Citrate | 0.6 | 1.68 |
| Talc | 8.1 | 22.37 |

*low viscosity with a methoxy content of 28-30% and hydroxypropyl content of 7-12%

### Example 2

A 60 mg multiparticulate duloxetine hydrochloride was prepared according to the procedure described in Example 1 except the delayed release coating was comprised a mixture of hypromellose phthalate (Shin-Etsu's HP-50) and hypromellose phthalate (Shin-Etsu's HP-55). The final 60 mg capsule had the following composition:

| Ingredient | % (w/w) | mg/capsule |
|---|---|---|
| SSNRI CORE | | |
| Duloxetine Hydrochloride | 26.9 | 67.35 |
| Sugar Spheres | 50.4 | 125.9 |
| Hypromellose* | 2.7 | 6.75 |
| SEAL COATING | | |
| Polyethylene Glycol | 2.0 | 5.0 |
| Hypromellose* | 2.0 | 5.0 |
| DELAYED RELEASE COATING | | |
| Hypromellose Phthalate (HP-50) | 8.0 | 20.16 |
| Hypromellose Phthalate (HP-55) | 2.0 | 5.04 |
| Acetyltributyl Citrate | 0.4 | 1.01 |
| Talc | 5.5 | 13.84 |

*low viscosity with a methoxy content of 28-30% and hydroxypropyl content of 7-12%

The 60 mg capsules described in Examples 1 and 2 were tested in vivo in a 3-way, single dose, crossover study with a commercially available 60 mg duloxetine hydrochloride product, CYMBALTA®, as the reference product. The testing was conducted according to the United States Food and Drug Administrations (FDA) procedures for measuring bioavailability and bioequivalence of an orally administered drug. A general description of the in vivo testing procedures can be found in the FDA documents entitled “Guidance for Industry-Bioavailability and Bioequivalence Studies for Orally Administered Drug Products-General Considerations” March 2003 and/or “Guidance for Industry-Food-Effect Bioavailability and Fed Bioequivalence Studies” December 2002 which are incorporated herein by reference. The data from the in vivo studies was analyzed using standard statistical procedures such as outlined in the FDA documents entitled “Statistical Procedures for Bioequivalence Studies Using a Standard Two-Treatment Crossover Design” July 1992 and/or “Statistical Approaches to Establishing Bioequivalence” which are incorporated herein by reference. The results of the in vivo testing are summarized in the following table:

| | | | |
|---|---|---|---|
| Study | Cymbalta ® | EXAMPLE 1 | EXAMPLE 2 |

FED (n = 13)

| | | | |
|---|---|---|---|
| AUC (ng/mL | 904.03 | 944.87 | 908.99 |
| hr) | (226.21-1867.12) | (322.40-2355.67) | (299.31-2144.35) |
| Cmax (ng/mL) | 55.64 | 48.47 | 49.31 |
| | (14.29-118.23) | (11.67-113.99) | (12.69-91.56) |
| Ln (AUC) ratio | n/a | 98.87 | 99.20 |
| Ln (Cmax) | n/a | 82.67 | 89.01 |
| ratio | | | |

FASTED (n = 16)

| | | | |
|---|---|---|---|
| AUC (ng/mL | 642.16 | 640.67 | 637.21 |
| hr) | (152.89-1598.62) | (130.43-1760.57) | (173.81-1651.19) |
| Cmax (ng/mL) | 39.98 | 39.75 | 38.67 |
| | (9.54-89.50) | (9.45-89.52) | (13.47-100.81) |
| Ln (AUC) ratio | n/a | 96.50 | 98.16 |
| Ln (Cmax) | n/a | 95.89 | 96.48 |
| ratio | | | |

The above data demonstrates a combination of enteric polymers in the delayed release coating allows the delayed release coating to be thinner, i.e. less total enteric polymer. The data also suggests that the use of a combination of enteric polymers in the delayed release coating improves the clinical properties of the dosage forms prepared in accordance with the present invention.

While certain preferred and alternative embodiments of the present invention have been set forth for purposes of disclosing the invention, modifications to the disclosed embodiments may occur to those who are skilled in the art. In addition, based upon the foregoing description and published literature, an individual of ordinary skill will understand that a pharmaceutical excipient can exhibit different properties depending upon the concentration in the dosage form or the manner in which it is formulated. For example, it is reported in the literature that microcrystalline cellulose can act as a tablet disintegrant at concentrations of 5-15% but as a tablet binder or diluent at concentrations of 20-90%. Accordingly, the appended claims are intended to cover all embodiments of the invention and modifications thereof which do not depart from the spirit and scope of the invention.

1. A multiparticulate oral pharmaceutical composition comprising a plurality of particles comprising:
(a) a core comprising: (i) a therapeutically effective amount of a selective serotonin and norepinephrine reuptake inhibitor and a pharmaceutically acceptable excipient;
(b) a seal coating that comprises only water soluble materials and wherein the seal coating is applied directly to the core; and
(c) a delayed release coating comprising an enteric material and at least one pharmaceutically acceptable processing aid and wherein the delayed release coating is applied directly to the seal coating.

2. The pharmaceutical composition as defined in claim 1 wherein the selective serotonin and norepinephrine reuptake inhibitor is selected from the group consisting of venlafaxine, desvenlafaxine, sibutramine, nefazodone, milnacipran, desipramine, duloxetine, bicifadine or pharmaceutically acceptable salts thereof.

3. The pharmaceutical composition as defined in claim 2 wherein the selective serotonin and norepinephrine reuptake inhibitor is duloxetine or pharmaceutically acceptable salts thereof.

4. The pharmaceutical composition as defined in claim 1 wherein the particles are delayed release coated granules, microtablets, beads or pellets.

5. The pharmaceutical composition as defined in claim 1 wherein the core comprises an inert starting seed coated with a drug layer comprising the selective serotonin and norepinephrine reuptake inhibitor and a binder.

6. The pharmaceutical composition as defined in claim 1 wherein the seal coating is a mixture of two water soluble polymers selected from the group consisting of hydroxypropyl methylcellulose, hydroxypropyl cellulose, polyvinylpyrrolidone, polyvinyl alcohol and polyethylene glycol.

7. The pharmaceutical composition as defined in claim 1 wherein the delayed release coating comprises the enteric material and a processing aid that is slightly soluble, very slightly soluble, insoluble or mixtures of the foregoing.

8. The pharmaceutical composition as defined in claim 7 wherein the delayed release coating consists of the enteric material and very slightly soluble or insoluble processing aids.

9. The pharmaceutical composition as defined in claim 8 wherein the processing aids are selected from the group consisting of plasticizers, lubricants, fillers, glidants, anti-adherents or mixtures of the foregoing.

10. The pharmaceutical composition as defined in claim 1 wherein the delayed release coating comprises the enteric material and an acetylalkyl citrate plasticizer.

11. The pharmaceutical composition as defined in claim 10 wherein the acetylalkyl citrate plasticizer is acetyltributyl citrate.

12. The pharmaceutical composition as defined in claim 1 wherein the delayed release coating comprises at least two enteric materials wherein each enteric material dissolves at a different pH.

13. The pharmaceutical composition as defined in claim 1 wherein the composition is a capsule.

14. The pharmaceutical composition as defined in claim 1 wherein the composition is a tablet.

15. A multiparticulate oral pharmaceutical composition comprising a plurality delayed release coated particles wherein the delayed release coated particle consists essentially of:
(a) a core comprising: (i) a therapeutically effective amount of duloxetine or a pharmaceutically acceptable salt thereof; (ii) a inert starting seed; and (iii) a water soluble binder;
(b) a seal coating that comprises only water soluble materials and wherein the seal coating is applied directly to the core; and
(c) a delayed release coating consisting essentially of an enteric material and slightly soluble, very slightly soluble or insoluble processing aids.

16. The pharmaceutical composition as defined in claim 15 wherein the delayed release coating comprises at least two enteric materials wherein each enteric material dissolves at a different pH.

17. The pharmaceutical composition as defined in claim 15 wherein the composition is a capsule.

18. The pharmaceutical composition as defined in claim 15 wherein the composition is a tablet.

19. The pharmaceutical composition as defined in claim 15 wherein the delayed release coating further consists of an acetylalkyl citrate plasticizer.

20. The pharmaceutical composition as defined in claim 19 wherein the acetylalkyl citrate plasticizer is acetyltributyl citrate.
